(19) United States
(12) Patent Application Publication
(10) Pub. No.: US 2004/0157810 A1
(43) Pub. Date: Aug. 12, 2004

(54) Corticosteroid conjugates and uses thereof

(76) Inventors: Martin H. Teicher, Rye, NH (US); Susan L. Andersen-Navalta, Medfield, MA (US)

Correspondence Address: CLARK & ELBING LLP, 101 FEDERAL STREET, BOSTON, MA, 02110, US

(21) Appl. No.: 10/646,063

(22) Filed: Aug. 22, 2003

Related U.S. Application Data
(60) Provisional application No. 60/405,688, filed on Aug. 23, 2002.

(51) Int. Cl. C07J 17/00; A61K 31/66; A61K 31/58

(52) U.S. Cl. 514/174; 514/125; 540/5; 540/114

(57) ABSTRACT

The invention features corticosteroids conjugated to either a charged group or a bulky group in a manner that resists in vivo cleavage, the resulting conjugate is a peripherally acting steroid with reduced activity in the central nervous system. The invention provides a method for treating a patient having an inflammatory disease by administering to the patient a corticosteroid conjugate.

### CROSS-REFERENCE TO RELATED APPLICATIONS

The application claims benefit of U.S. Provisional Application No. 60/405,688, filed Aug. 23, 2002, which is incorporated herein by reference.

### BACKGROUND OF THE INVENTION

The invention relates to the field of corticosteroids.

The mineralocorticoid, aldosterone, and the glucocorticoids, cortisol and orticosterone, are produced in the adrenal cortex. These steroids act by binding to receptors which then act to modulate gene transcription in target tissues.

Corticosteroids are used to treat swelling, redness, itching, allergic reactions, and a wide range of conditions including: allergic rhinitis, ankylosing spondylitis, asthma, atopic dermatitis, autoimmune disorders, bursitis, Crohn's disease, congenital adrenal hyperplasia, contact dermatitis, dermatological disorders, drug hypersensitivity reactions, endocrine disorders, hypercalcemia associated with cancer, iritis and iridocyclitis, nonsuppurative thyroiditis; primary or secondary adrenocortical insufficiency, psoriatic and rheumatoid arthritis, tendinitis and non-specific tenosynovitis, and ulcerative colitis.

The brain is well protected from outside influences by the blood-brain barrier, which prevents the free entry of many circulating molecules, cells or micro-organisms into the brain interstitial space. However, this is not true for corticosteroids, which penetrate the blood-brain barrier. Thus, in the treatment of peripheral disorders (e.g., asthma or arthritis), the brain is exposed to the corticosteroid without any therapeutic benefit and with the possibility of severe adverse effects. These adverse effects, which are described in the PDR, include: insomnia, euphoria, mood changes, nervousness, personality changes, depression, severe nausea, headaches, and convulsions.

Even topical and ocular administration of corticosteroids can enter the systemic circulation, cross the blood-brain-barrier, and effect the regulation of the hypothalamic-pituitary-adrenal axis (see, for example, Krupin et al., Arch Ophthalmol, 94:919-20 (1976) and Meredig et al., Klin MonatsblAugenheilkd, 176:907-10 (1980)). Thus, even topical administration of corticosteroids for the treatment of chronic conditions can have untoward CNS effects.

21-phosphate and 21-succinate esters of corticosteroids, which are clinically well known, are modified by inclusion of a charged moiety to render them soluble in aqueous solutions for intravenous injection. Both 21-phosphate and 21-succinate esters of corticosteroids are rapidly hydrolyzed in the bloodstream to produce the free steroid (see, for example, Miyabo et al., Eur J Clin. Pharmacol, 20:277-82 (1981)). These modifications do not reduce CNS activity. For example, dexamethasone sodium phosphate is CNS active, producing suppression of cortisol production or ACTH (see, Abou Samra et al., J. Clin. Endocrinol. Metab., 6 1:116-9 (1985), Kemppainen R. J., and Sartin J. L., Am. J. Vet. Res., 45:742-6 (1984), Linquette et al., Pathol. Biol. (Paris), 28:85-9 (1980), and Petersen et al., Fur. J. Clin. Pharmacol., 25:643-50 (1983)). Hydrocortisone sodium succinate is also CNS active (see Kasperlik-Zaluska et al., Horm. Metab. Res., 12:676-9 (1980) and Posener et al., Psychoneuro-endocrinology, 22:169-76 (1997)). Because these compounds are designed to be rapidly cleaved in vivo, producing their parents dexamethasone and hydrocortisone, respectively, no reduction in CNS activity is achieved.

### SUMMARY OF THE INVENTION

We have discovered that when a corticosteroid is conjugated to either a charged group or a bulky group in a manner that resists in vivo cleavage, the resulting conjugate is a peripherally acting steroid with reduced activity in the central nervous system. The invention provides structurally modified corticosteroids with altered biodistributions, thereby reducing the occurrence of adverse reactions associated with this class of drug.

The invention features a corticosteroid conjugate comprising a corticosteroid covalently attached via a linker to a bulky group of greater than 400 daltons or a charged group of less than 400 daltons. The corticosteroid conjugate has anti-inflammatory activity in vivo and reduced activity in the central nervous system in comparison to the parent corticosteroid.

The corticosteroid conjugate is further described by formula I:

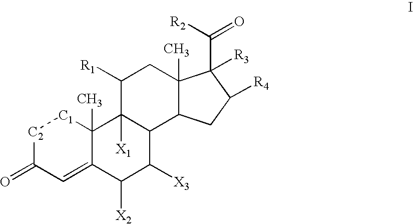

In formula I, the bond between C₁ and C₂ is a double or a single bond; X₁ represents H or a halogen atom; X₂ represents H, CH₃, or a halogen atom; X₃ represents H or a halogen atom; R₁ represents ═O or —OH; R₂ represents CH₃, SCH₂F, CH₂Cl, CH₂G¹, CH₂OH, CH₂O—P(O)(O⁻)₂, CH₂O-acyl, CH₂NHG¹, CH₂SG¹, or CH₂OG¹; R₃ and R₄ each independently represent H, C₁₋₁₀ alkyl, —OH, —O-acyl, —OR, or R₃ and R₄ combine to form a cyclic acetal described by formula II:

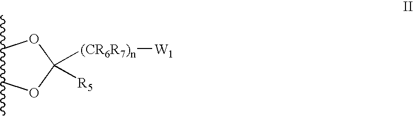

where n is a whole integer from 0 to 6; R₅, R₆, and R₇ each independently represent H or C₁₋₁₀ alkyl; W₁ represents H, CH₃, G¹, NR₈G¹, OG¹, SG¹, —NH—NH-G¹, C(O)-G¹, or C(S)-G¹; R₈ is H, C₁₋₁₀ alkyl or C₅₋₁₀ aryl; and G¹ is a bond between the corticosteroid and the linker.

Desirably, linker L is described by formula III:

G¹-(Z¹)ₒ-(Y¹)-(Z²)ₛ-(R₁₀)-(Z³)ₜ-(Y²)ᵥ-(Z⁴)ₚ-G² III

In formula III, G¹ is a bond between the corticosteroid and the linker, G² is a bond between the linker and the bulky group or between the linker and the charged group, each of Z¹, Z², Z³, and Z⁴ is, independently, selected from O, S, and NR₁₁; R₁₁ is hydrogen or a C₁₋₁₀ alkyl group; each of Y¹ and Y² is, independently, selected from carbonyl, thiocarbonyl, sulphonyl, phosphoryl or similar acid-forming groups; o, p, s, t, u, and v are each independently 0 or 1; and R₁₀ is a C₁₋₁₀ alkyl, a linear or branched heteroalkyl of 1 to 10 atoms, a C₂₋₁₀ alkene, a C₂₋₁₀ alkyne, a C₅₋₁₀ aryl, a cyclic system of 3 to 10 atoms, —(CH₂CH₂O)_{q}CH₂CH₂— in which q is an integer of 1 to 4, or a chemical bond linking G¹-(Z¹)ₒ-(Y¹)ᵤ-(Z²)ₛ- to -(Z³)ₜ-(Y²)ᵥ-(Z⁴)ₚ-G².

The bulky group can be a naturally occurring polymer or a synthetic polymer. Examples of natural polymers that can be used include, without limitation, glycoproteins, polypeptides, or polysaccharides. Desirably, when the bulky group includes a natural polymer, the natural polymer is selected from alpha-1-acid glycoprotein and hyaluronic acid. Examples of synthetic polymers that can be used as bulky groups include, without limitation, polyethylene glycol, and the synthetic polypetide N-hxg. The bulky group may also include another corticosteroid.

The charged group can be a cation or an anion. Desirably, the charged group is a polyanion including at least three negatively charged moieties or a cation having at least one positively charged moiety.

The corticosteroid conjugates of the invention may be used to treat inflammatory conditions, including conditions resulting from an immune response in a mammal. Thus, the invention features a method of treating or preventing an autoimmune or inflammatory condition in a mammal by administering to the mammal an effective amount of one or more corticosteroid conjugates of the invention. The conditions to be treated using the methods of the invention include, without limitation, asthma, psoriasis, eczema, organ/tissue transplant rejection, graft versus host reactions, Raynaud's syndrome, autoimmune thyroiditis, Grave's disease, autoimmune hemolytic anemia, autoimmune thromboeytopenia purpura, mixed connective tissue disease, idiopathic Addison's disease, Sjogren's syndrome, urticaria, dermatitis, multiple sclerosis, rheumatoid arthritis, insulin-dependent diabetes mellitus, uveitis, Crohn's disease, ulcerative colitis, lupus, tendonitis, bursitis, adult respiratory distress syndrome, shock, oxygen toxicity, glomerulonephritis, vasculitis, reactive arthritis, necrotizing enterocolitis, Goodpasture's syndrome, hypersensitivity pneumonitis, glomerulonephritis; encephalomyelitis, and meningitis.

The invention features a method for inhibiting passage across the blood-brain barrier of a corticosteroid by covalent attachment of a group, the group being a bulky group of greater than 400 daltons or a charged group of less than 400 daltons. The group increases the size, or alters the charge, of the corticosteroid sufficiently to inhibit passage across the blood-brain barrier without destroying the anti-inflammatory activity of the corticosteroid covalently attached to the group. Desirably, the covalent attachment is resistant to in vivo cleavage, further protecting the brain from CNS active metabolites. The bulky group or charged group charged can be attached to the corticosteroid through any of positions C16, C17, or C21 of the corticosteroid.

The invention features a pharmaceutical composition that includes an effective amount of a corticosteroid conjugate described herein in any pharmaceutically acceptable form, along with a pharmaceutically acceptable carrier or diluent.

By “C₁₋₁₀ alkyl” is meant a branched or unbranched saturated hydrocarbon group, having 1 to 10 carbon atoms, inclusive. An alkyl may optionally include monocyclic, bicyclic, or tricyclic rings, in which each ring desirably has three to six members. The alkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₂₋₁₀ alkene” is meant a branched or unbranched hydrocarbon group containing one or more double bonds, desirably having from 2 to 10 carbon atoms. A C₂₋₁₀ alkene may optionally include monocyclic, bicyclic, or tricyclic rings, in which each ring desirably has five or six members. The C₂₋₁₀ alkene group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₂₋₁₀ alkyne” is meant a branched or unbranched hydrocarbon group containing one or more triple bonds, desirably having from 2 to 10 carbon atoms. A C₂₋₁₀ alkyne may optionally include monocyclic, bicyclic, or tricyclic rings, in which each ring desirably has five or six members. The C₂₋₁₀ alkyne group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “heteroalkyl” is meant a branched or unbranched alkyl group in which one or more methylenes (—CH₂—) are replaced by nitrogen, oxygen, sulfur, carbonyl, thiocarbonyl, phosphoryl, or sulfonyl moieties. Some examples include tertiary amines, ethers, thioethers, amides, thioamides, carbamates, thiocarbamates, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups

By “C₅₋₁₀ aryl” or “aryl” is meant an aromatic group having a ring system with conjugated π electrons (e.g., phenyl, or imidazole). The ring of the aryl group is preferably 5 to 10 atoms. The aromatic ring may be exclusively composed of carbon atoms or may be composed of a mixture of carbon atoms and heteroatoms. Preferred heteroatoms include nitrogen, oxygen, sulfur, and phosphorous. Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

The term “cyclic system” refers to a compound that contains one or more covalently closed ring structures, in which the atoms forming the backbone of the ring are composed of any combination of the following: carbon, oxygen, nitrogen, sulfur, and phosphorous. The cyclic system may be substituted or unsubstituted. Exemplary substituents include, without limitation, alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

By “cyclic acetal” is meant a ring structure including two oxygen atoms separated by a carbon atom which is optionally substituted (e.g., 1,3-dioxolane). Exemplary substituents include, without limitation, alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, quaternary amino, phosphodiester, phosphoramidate, phosphate, phosphonate, phosphonate ester, sulfonate, sulfate, sulfhydryl, phenol, amidine, guanidine, and imidazole groups.

By “acyl” is meant is meant a chemical moiety with the formula —C(O)R′, where R′ is selected from the group consisting of C₁₋₁₀ alkyl, C₂₋₁₀ alkene, heteroalkyl, C₂₋₁₀ alkyne, C₅₋₁₀ aryl, and cyclic system. Examples of acyl groups include, without limitation, acetyl, propanoyl, butanoyl, pentanoyl, and tetrahydrofuran-2-oyl.

By “fluoroalkyl” is meant an alkyl group that is substituted with a fluorine.

By “perfluoroalkyl” is meant an alkyl group consisting of only carbon and fluorine atoms.

By “carboxyalkyl” is meant a chemical moiety with the formula —(R)—COOH, wherein R is an alkyl group.

By “hydroxyalkyl” is meant a chemical moiety with the formula —(R)—OH, wherein R is an alkyl group.

By “alkoxy” is meant a chemical substituent of the formula —OR, wherein R is an alkyl group.

By “aryloxy” is meant a chemical substituent of the formula —OR, wherein R is a C₅₋₁₀ aryl group.

By “alkylthio” is meant a chemical substituent of the formula —SR, wherein R is an alkyl group.

By “arylthio” is meant a chemical substituent of the formula —SR, wherein R is a C₅₋₁₀ aryl group.

By “quaternary amino” is meant a chemical substituent of the formula —(R)—N(R′)(R″)(R′″)⁺, wherein R, R′, R″, and R′″ are each independently a C₁₋₁₀ alkyl, C₂₋₁₀ alkene, C₂₋₁₀ alkyne, or C₅₋₁₀ aryl. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve the patient's condition. Thus, in the claims and embodiments, treating is the administration to a mammal either for therapeutic or prophylactic purposes.

The term “administration” or “administering” refers to a method of giving a dosage of a pharmaceutical composition to a mammal, wherein the corticosteroid conjugate is administered by a route selected from, without limitation, inhalation, ocular administration, nasal instillation, parenteral administration, dermal administration, transdermal administration, buccal administration, rectal administration, sublingual administration, perilingual administration, nasal administration, topical administration and oral administration. Parenteral administration includes intravenous, intraperitoneal, subcutaneous, and intramuscular administration. The preferred method of administration can vary depending on various factors, e.g., the components of the pharmaceutical composition, site of the potential or actual disease and severity of disease.

The term “mammal” includes, without limitation, humans, cattle, pigs, sheep, horses, dogs, and cats.

By “parent corticosteroid” is meant the corticosteroid which is modified by conjugation to a bulky group or a charged group.

By “reduced CNS activity” for a corticosteroid conjugate is meant that the ratio of AUC_{brain} (area under the curve in brain tissue) to AUC_{blood} (area under the curves in whole blood) is reduced for the corticosteroid conjugate in comparison to the parent corticosteroid administered under the same conditions. The AUC calculation includes the administered compound and any metabolites, having anti-inflammatory activity, thereof.

By “resistant to in vivo cleavage” is meant that, in vivo, less than 30, 20, 10, 5, 2, or 1 percent of the administered drug is cleaved, separating the corticosteroid from the charged group or the bulky group, prior to excretion.

By “linked through positions C16, C17, and/or C21” is meant that the charged group, bulky group, or linker is covalently attached to a substituent of positions C16, C17, and/or C21 as identified by the numbering scheme shown below. For any reference provided herein to a numbered position in a corticosteroid, the recited position is defined by the numbering scheme below.

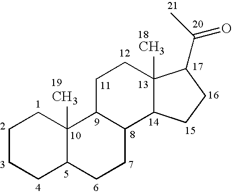

By “charged moiety” is meant a moiety which loses a proton at physiological pH thereby becoming negatively charged (e.g., carboxylate, or phosphodiester), a moiety which gains a proton at physiological pH thereby becoming positively charged (e.g., ammonium, guanidinium, or amidinium), a moiety that includes a net formal positive charge without protonation (e.g., quaternary ammonium), or a moiety that includes a net formal negative charge without loss of a proton (e.g., borate, BR₄⁻).

### DETAILED DESCRIPTION

The invention features peripherally acting corticosteroid conjugates which have reduced CNS activity in comparison their parent corticosteroids. The corticosteroid conjugates described herein have three characteristic components: a corticosteroid covalently tethered, via a linker, to a group that is bulky or charged.

Corticosteroids

Corticosteroids which can be modified to inhibit passage across the blood-brain barrier include, without limitation, hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flunisolide, flucinolone acetonide, fluocinonide, fluorocinolone acetonide, fluocortin butyl, fluocortolone, fluorocortolone hexanoate, diflucortolone valerate, fluorometholone, fluperolone acetate, fluprednidene acetate, fluprednisolone, flurandenolide, formocortal, halcinonide, halometasone, halopredone acetate, hydrocortamate, hydrocortisone, hydrocortisone acetate, hydrocortisone butyrate, hydrocortisone phosphate, hydrocortisone 21-sodium succinate, hydrocortisone tebutate, mazipredone, medrysone, meprednisone, methylprednicolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar anti-inflammatory properties are also intended to be encompassed by this group.

The structures of several of the above-mentioned corticosteroids are provided in Table 1. These are structural examples of parent corticosteroids which can be modified as described herein to achieve a reduction in CNS activity. Corticosteroid conjugates of the invention are prepared by modification of an available functional group present in the parent corticosteroid. Alternatively, an acyl or cyclic acetal group can be removed from the parent corticosteroid prior to conjugation with a bulky group or a charged group.

TABLE 1 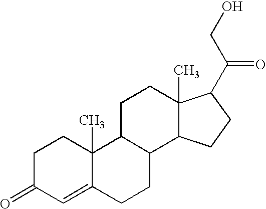
desoxycorticosterone 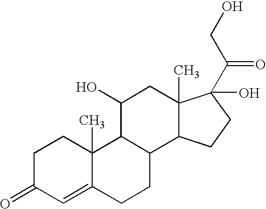
hydrocortisone 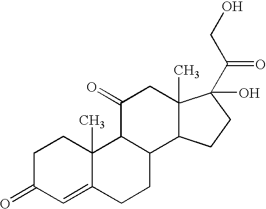
cortisone 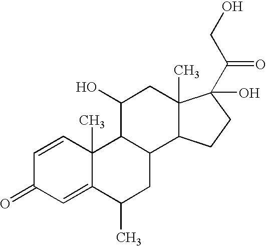
methylprednisolone 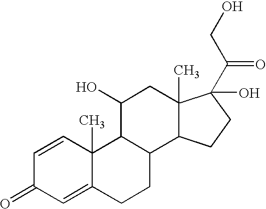
prednisolone 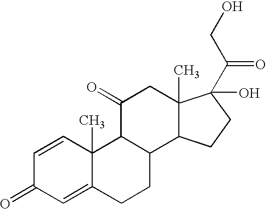
prednisone 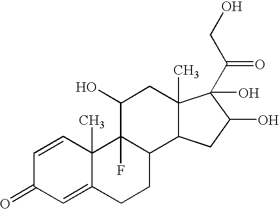
triamcinolone 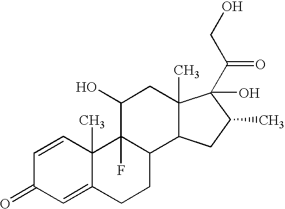
dexamethasone 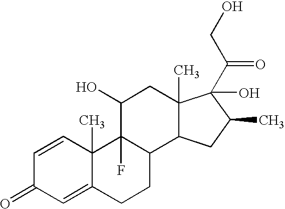
betamethasone 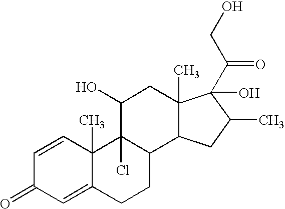
beclomethasone 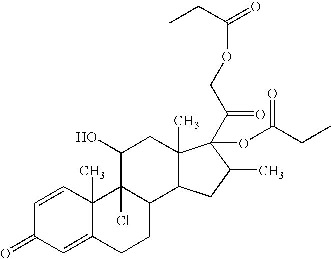
beclomethasone- 17,21-diproprionate 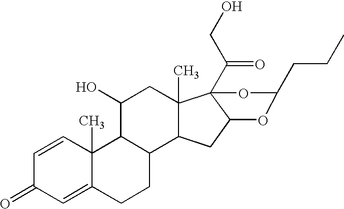
budesonide 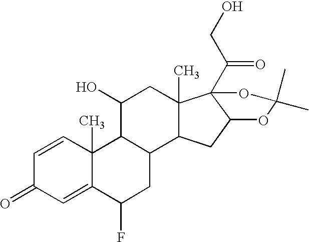
flunisolide 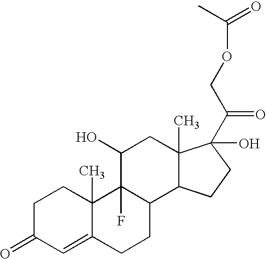
fludrocortisone 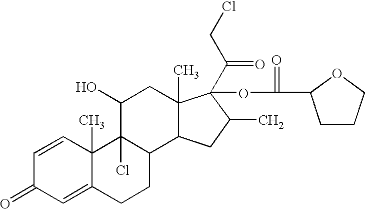
mometasone 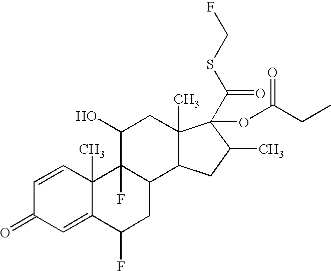
fluticasone 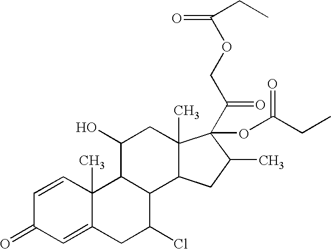
alclometasone 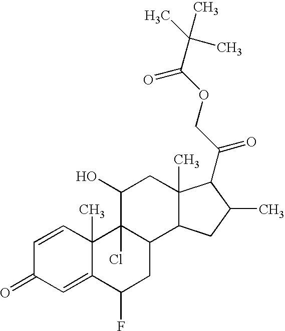
clocortolone 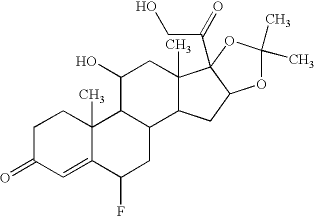
flurandrenolide 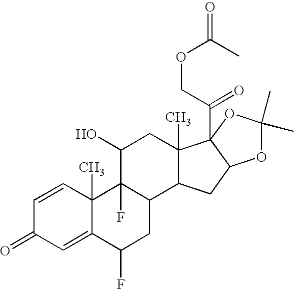
fluocinonide 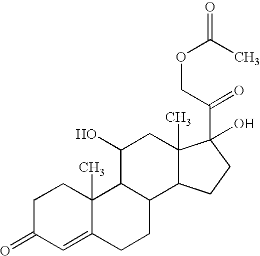
hydrocortisone acetate 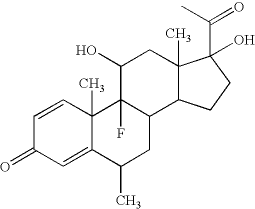
fluorometholone 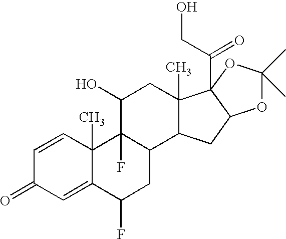
fluocinolone acetonide 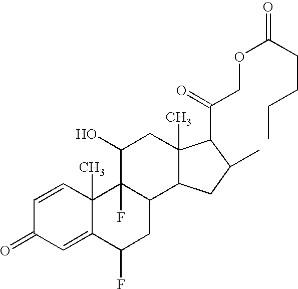
diflucortolone valerate 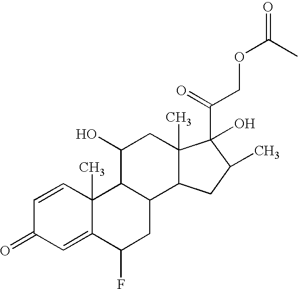
paramethasone acetate 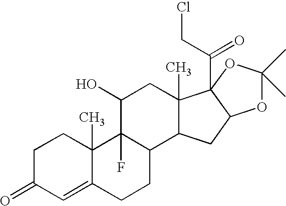
halcinonide 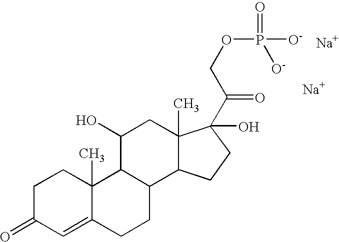
hydrocortisone phosphate 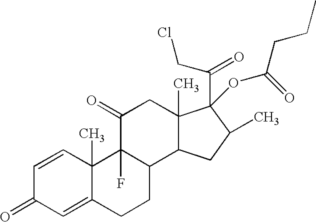
clobetasone butyrate 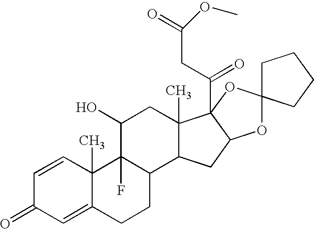
amcinonide 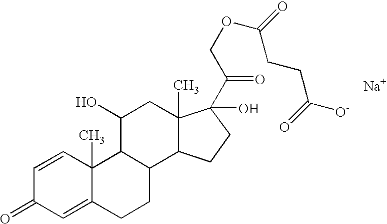
prednisolone succinate

Linkers

The linker component of the invention is, at its simplest, a bond between a corticosteroid and a group that is bulky or charged. The linker provides a linear, cyclic, or branched molecular skeleton having pendant groups covalently linking a corticosteroid to a group that is bulky or charged.

Thus, the linking of a corticosteroid to a group that is bulky or charged is achieved by covalent means, involving bond formation with one or more functional groups located on the corticosteroid and the bulky or charged group. Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of a corticosteroid and a group that is bulky or charged may be effected using a linker which contains reactive moieties capable of reaction with such functional groups present in the corticosteroid and the bulky or charged group. For example, a hydroxyl group of the corticosteroid may react with a carboxyl group of the linker, or an activated derivative thereof, resulting in the formation of an ester linking the two.

Examples of moieties capable of reaction with sulfhydryl groups include α-haloacetyl compounds of the type XCH₂CO— (where X=Br, Cl or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulphide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X=Cl, Br or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g. wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962). Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in the corticosteroid and/or the bulky or charged group may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate. When the C16 and C17 positions of the corticosteroid both have hydroxy substituents, these hydroxy groups, together a vicinal diol, can be converted into a cyclic acetal as described by, for example, J. March, Advanced Organic Chemistry: Reactions, Mechanisms and Structure, John Wiley & Sons, Inc. pp. 889-890, 1992. The acetal can include a reactive group (e.g., an amino or carboxyl group) capable of forming a bond with a bulky or charged group.

So-called zero-length linkers, involving direct covalent joining of a reactive chemical group of the corticosteroid with a reactive chemical group of the bulky or charged group without introducing additional linking material may, if desired, be used in accordance with the invention. For example, the amino group at C21 in a 21-amino corticosteroid can be converted to a guanidine group as described in Example 8. The resulting guanidine derivative is a cation at physiological pH.

Most commonly, however, the linker will include two or more reactive moieties, as described above, connected by a spacer element. The presence of such a spacer permits bifunctional linkers to react with specific functional groups within the corticosteroid and the bulky or charged group, resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between the corticosteroid and the bulky or charged group.

Spacer elements in the linker typically consist of linear or branched chains and may include a C₁₋₁₀ alkyl, a heteroalkyl of 1 to 10 atoms, a C₂₋₁₀ alkene, a C₂₋₁₀ alkyne, C₅₋₁₀ aryl, a cyclic system of 3 to 10 atoms, or —CH₂CH₂O)ₙCH₂CH₂—, in which n is 1 to 4.

In some instances, the linker is described by formula III:

G¹-(Z¹)ₒ-(Y¹)ᵤ-(Z²)ₛ-(R₁₀)-(Z³)ₜ(Y²)ᵥ-(Z⁴)ₚ-G² III

In formula III, G¹ is a bond between the corticosteroid and the linker, G² is a bond between the linker and the bulky group or between the linker and the charged group, each of Z¹, Z², Z³, and Z⁴ is, independently, selected from O, S, and NR₁₁; R₁₁ is hydrogen or a C₁₋₁₀ alkyl group; each of Y¹ and Y² is, independently, selected from carbonyl, thiocarbonyl, sulphonyl, phosphoryl or similar acid-forming groups; o, p, s, t, u, and v are each independently 0 or 1; and R₁₀ is a C₁₋₁₀ alkyl, a linear or branched heteroalkyl of 1 to 10 atoms, C₂₋₁₀ alkene, a C₂₋₁₀ alkyne, a C₅₋₁₀ aryl, a cyclic system of 3 to 10 atoms, —(CH₂CH₂O)_{q}CH₂CH₂—in which q is an integer of 1 to 4, or a chemical bond linking G¹-(Z¹)ₒ-(Y¹)ᵤ-(Z²)ₛ- to -(Z³)ₜ-(Y²)ᵥ-(Z⁴)ₚ-G².

Bulky Groups

The function of the bulky group is to increase the size of the corticosteroid sufficiently to inhibit passage across the blood-brain barrier. Bulky groups capable of inhibiting passage of the corticosteroid across the blood-brain barrier include those having a molecular weight greater than 400, 500, 600, 700, 800, 900, or 1000 daltons. Desirably, these groups are attached through one or more of the C16, C17, and C21 positions of the corticosteroid.

The bulky group may include one or more additional corticosteroids, the corticosteroids can be linked as dimers, trimers, or tetramers, as shown below, where each corticosteroid (A) is the same or different within each corticosteroid conjugate.

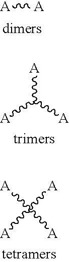

The bulky group may also be charged. For example, bulky groups include, without limitation, charged polypeptides, such as poly-arginine (guanidinium side chain), poly-lysine (ammonium side chain), poly-aspartic acid (carboxylate side chain), poly-glutamic acid (carboxlyate side chain), or poly-histidine (imidazolium side chain). An exemplary charged polysaccharide is hyaluronic acid (see below).

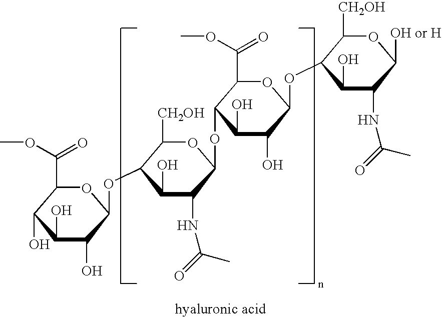

Desirably, a bulky group is selected which enhances the cellular uptake of the conjugate. For example, certain peptides enable active translocation across the plasma membrane into cells (e.g., RKKRRQRRR, the Tat(49-57) peptide). Exemplary peptides which promote cellular uptake are disclosed, for example, by Wender et al., Natl Acad Sci USA 97(24):13003-8 (2000) and Laurent et al., FEBS Lett 443(1):61-5 (1999), incorporated herein by reference. An example of a charged bulky group which facilitates cellular uptake is the polyguanidine peptoid (N-hxg)₉, shown below. Each of the nine guanidine side chains is a charged guanidinium cation at physiological pH.

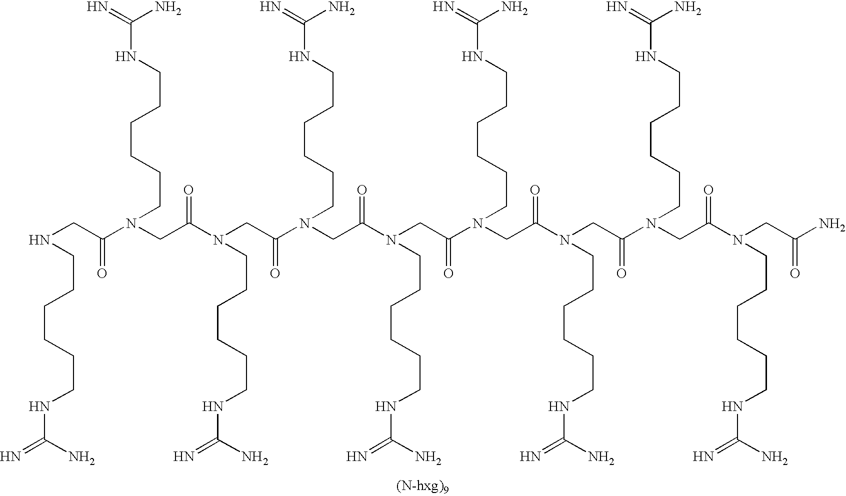

Charged Groups

The function of the charged group is to alter the charge of the corticosteroid sufficiently to inhibit passage across the blood-brain barrier. Desirably, charged groups are attached through one or more of the C16, C17, and C21 positions of the corticosteroid.

A charged group may be cationic or an anionic. Charged groups include 2, 3, 4, 5, 6, 7, 8, 9, 10, or more negatively charged moieties or 1, 2, 3, 4, 5, 6, 7, 8, 9, 10, or more positively charged moieties. Charged moieties include, without limitation, carboxylate, phosphodiester, phosphoramidate, borate, phosphate, phosphonate, phosphonate ester, sulfonate, sulfate, thiolate, phenolate, ammonium, amidinium, guanidinium, quaternary ammonium, and imidazolium moieties.

Corticosteroid Conjugates

The corticosteroid conjugates of the present invention are designed to largely remain intact in vivo, resisting cleavage by intracellular and extracellular enzymes (e.g., amidases, esterases, and phosphatases). Any in vivo cleavage of the corticosteroid conjugate produces the parent steroid, resulting in the unnecessary and potentially harmful exposure of the central nervous system to this corticosteroid. Thus, the corticosteroid conjugates of the invention are not prodrugs, but are therapeutically active in their conjugated form, resulting in an improved therapeutic index relative to their parent, unconjugated, corticosteroid.

Corticosteroid conjugates are further described by any one of formulas IV-VIII:

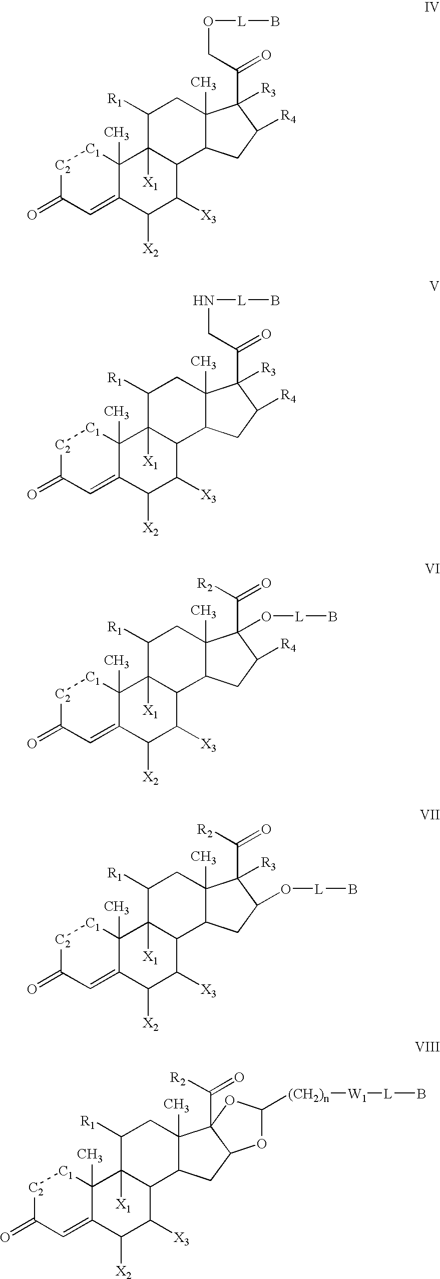

In formulas IV-VIII, the bond between C₁ and C₂, X₁, X₂, X₃, R₁, R₂, R₃, R₄, and W₁ are as described above. L is a linker of formula III, described above. B is a bulky or charged group, as described above.

Corticosteroid conjugates can be prepared using techniques familiar to those skilled in the art. The conjugates can be prepared using the methods disclosed in, for example, G. Hermanson, Bioconjugate Techniques, Academic Press, Inc., 1996, as well as U.S. Pat. Nos. 2,779,775, 2,932,657, 4,472,392, 4,609,496, 4,820,700, 4,948,533, 4,950,659, 5,063,222, 5,215,979, 5,482,934, 5,939,409, and 6,140,308, each of which is incorporated herein by reference. Additional synthetic details are provided in Examples 1-8.

Assays

Corticosteroid conjugates can be assayed by using standard in vitro models or animal models to evaluate their therapeutic activity. These assays are presently described in the literature and are familiar to those skilled in the art. Some of these are described below and in the Examples.

The biodistribution of a corticosteroid conjugate can be measured by autoradiography. (see Example 9).

The cytoplasmic binding of a corticosteroid conjugate can be ascertained by displacement binding (see Example 10).

The dose-dependent capacity of corticosteroid conjugates to suppress production of corticosterone in intact rats can be measured (see Example 11). Corticosterone levels are regulated by a feedback circuit that includes the pituitary gland, hypothalamus and higher brain centers, most notably the hippocampus. The capacity of synthetic glucocorticoids to induce feedback inhibition of cortisol production is significantly affected by the binding of the synthetic glucocorticoid to receptors in the hypothalamus (see, for example, Kovacs K. J. and Makara G. B. Brain Res., 474:205-10 (1988) and Sakakura et al., Neuroendocrinology, 32:174-8 (1981)) and possibly the hippocampus (see, for example, Sapolsky et al., Neuroendocrinology 51:328-36 (1990)). The hypothalamus and hippocampus have tight blood-brain-barriers, while the pituitary gland has a leaky or permeable barrier (see, Ruhle et al., Neuropeptides 22:117-24(1992)). Hence, synthetic glucocorticoids with reduced CNS activity should be less effective than the highly permeable parent corticosteroid in suppressing corticosterone production. The lowest dose of dexamethasone fully effective in suppressing basal corticosterone production for 24 hours was 0.025 mg/kg (Lurie et al., Biol. Psychiatry 26:26-34(1989)). This dose also significantly suppressed ether-stress induced increase in corticosterone, but a higher dose was necessary to fully eliminate response to this stressor.

The neurotoxic effects of corticosteroid conjugates can be assessed using OX42 immunohistochemistry (see Example 12). Failure to observe a dose-dependent effect of the corticosteroid conjugates would indicate that they do not cross the blood-brain-barrier to a sufficient extent to induce damage to neuronal populations. A rightward shift in the dose response curve indicated by a higher ED₅₀ would indicate partial protection (i.e. there is reduced CNS activity). Desirable corticosteroid conjugates have an ED₅₀ of at least 10-fold higher than their parent corticosteroids.

To establish the systemic efficacy of corticosteroid conjugates, their binding to glucocorticoid and mineralocorticoid receptors can be assayed in cytosol from thymus, fibroblasts, and kidney (see, Example 13).

The effects of corticosteroid conjugates on the liver will be determined in adrenalectomized male rats using the method described by Vicent et al., Mol. Pharmacol., 52:749-53 (1997) (see Example 14). Effective glucocorticoids produce a marked increase in liver glycogen accumulation.

The effects of corticosteroid conjugates on the thymus will be assessed in male Sprague-Dawley rats (see Example 15). Effective glucocorticoids will induce marked involution of the thymus gland.

Therapy

Corticosteroid conjugates can be administered locally or systemically to decrease inflammatory and immune responses. They can be used systemically in high doses in emergencies for anaphylactic reactions, spinal chord trauma, or shock. They can used in lower doses to treat allergic reactions such as heaves, hives, itching, and inflammatory diseases including arthritis.

Therapeutic formulations may be in the form of liquid solutions or suspensions; for oral administration, formulations may be in the form of tablets or capsules; for ocular administration, formulations may be in the form of eye drops; for topical administration, formulations may be in the form of creams or lotions; and for intranasal formulations, in the form of powders, nasal drops, or aerosols.

Methods well known in the art for making formulations are found, for example, in “Remington: The Science and Practice of Pharmacy” (20th ed., ed. A. R. Gennaro AR., 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

Corticosteroid conjugates may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, calcium, sodium, potassium and the like.

Administration of corticosteroid conjugates in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release of the corticosteroid conjugate. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Pharmaceutical formulations of the corticosteroid conjugates described herein include isomers such as diastereomers and enantiomers, mixtures of isomers, including racemic mixtures, salts, solvates, and polymorphs thereof.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1

### Protection and Deprotection of Reactive Groups

The synthesis of corticosteroid conjugates may involve the selective protection and deprotection of alcohols, amines, ketones, sulfhydryls or carboxyl functional groups of the corticosteroid, the linker, the bulky group, and/or the charged group. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. An acetal can be used to protect a ketone (═O) at the C3 and/or C11 positions of a corticosteroid using the methods described in, for example, U.S. Pat. No. 2,779,775. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994.

In the examples that follow, the use of protecting groups is indicated in a structure by the letter P, where P for any amine, aldehyde, ketone, carboxyl, sulfhydryl, or alcohol may be any of the protecting groups listed above.

### Example 2

### Preparation of C21 Derivatives of Prednisolone

21-methanesulfonate prednisolone can be prepared according to the methods described in U.S. Pat. No. 2,932,657. The corresponding amine can be prepared by reaction with potassium phthalimide followed by hydrolysis as described by, for example, J. March, Advanced Organic Chemistry: Reactions, Mechanisms and Structure, John Wiley & Sons, Inc. page 426, 1992. The free amine of the 21-amino prednisolone derivative can be reacted with an activated carboxyl. Carboxyls can be activated, for example, by formation of an active ester, such as nitrophenylesters, N-hydroxysuccinimidyl esters, or others as described in Chem. Soc. Rev. 12:129, 1983 and Angew. Chem. Int. Ed. Engl. 17:569, 1978, incorporated herein by reference. For example, oxalic acid (Aldrich, catalogue number 24,117-2) can be attached as a linking group, as shown below in reaction 1.

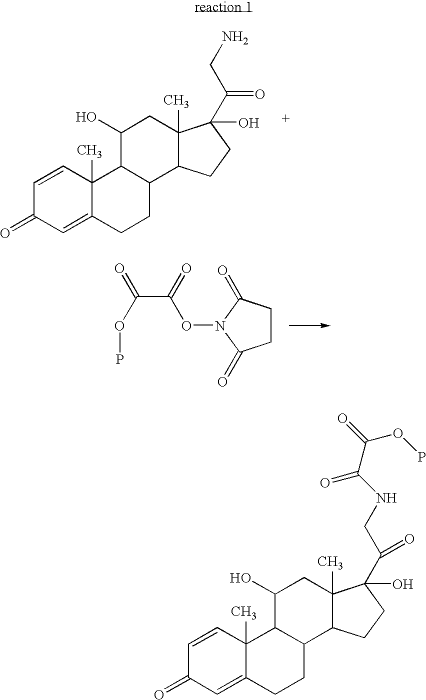

The protecting group in the reaction product can be removed by hydrolysis. The resulting acid is available for conjugation to a bulky group or a charged group.

### Example 3

### Preparation of a Polyguanidine Peptoid Derivative of Prednisolone

The polyguanidine peptoid N-hxg, shown below, can be prepared according to the methods described by Wender et al., Natl Acad Sci USA 97(24):13003-8, 2000, incorporated herein by reference.

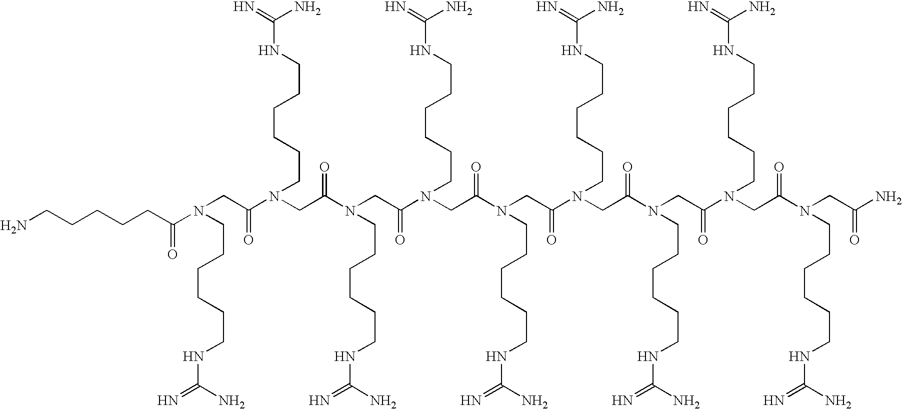

### N-hxg with an aminohexanoic acid linker at the N-terminus

The carboxyl derivative of prednisolone from Example 2 can be activated, vide supra, and conjugated to the protected precursor of N-hxg followed by the formation of the guanidine moieties and cleavage from the solid phase resin, as described by Wender ibid., to produce the polyguanidine prednisolone conjugate shown below.

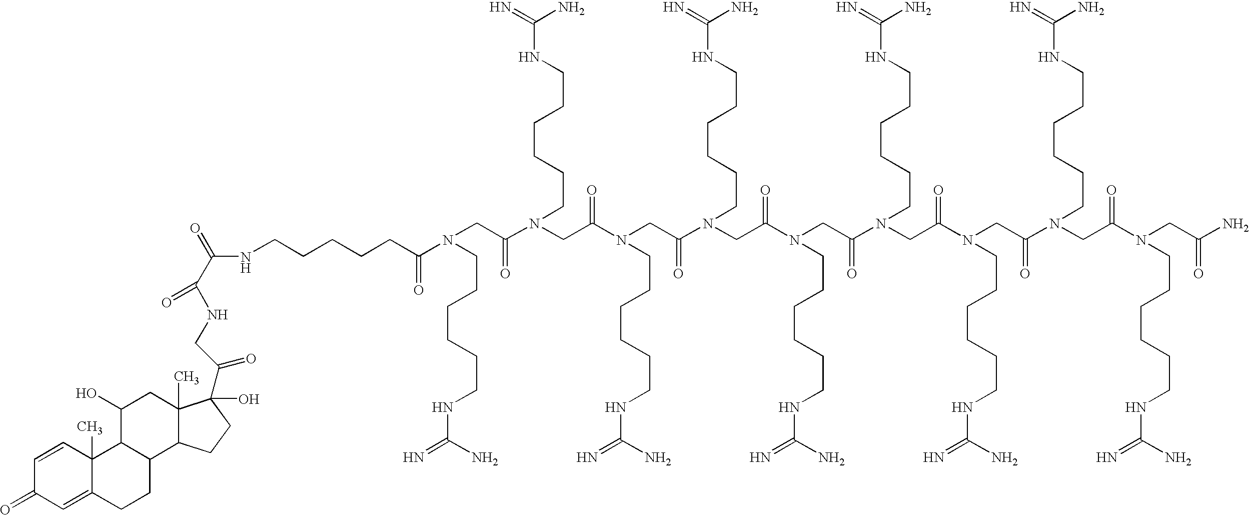

In this example, the bulky group has a molecular weight of over 1900 Daltons. Accordingly, in the example above, prednisolone is conjugated to a bulky group containing several positively charged moieties.

### Example 4

### Preparation of C16-C17 Cyclic Acetals of Triamcinolone

The cyclic acetal of triamcinolone can be prepared by the methods disclosed in U.S. Pat. No. 5,482,934, incorporated herein by reference. First the hydroxy groups at positions C16, C17, and C21 are acetylated by reaction with acetic anhydride, reaction 2 below.

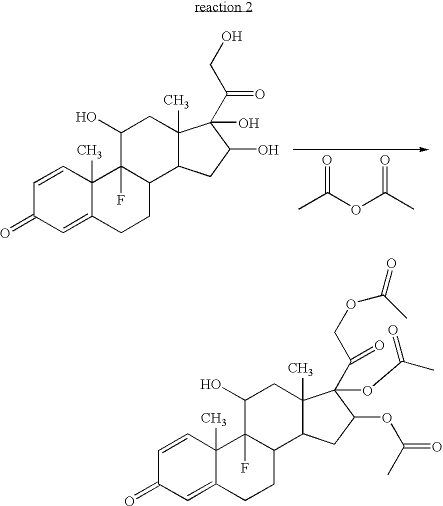

The esters at C16 and C17 are selectively removed by hydrolysis with hydrochloric acid and the resulting hydroxyl groups reacted with an appropriately substituted aldehyde to form the corresponding cyclic acetal as shown in reaction 3.

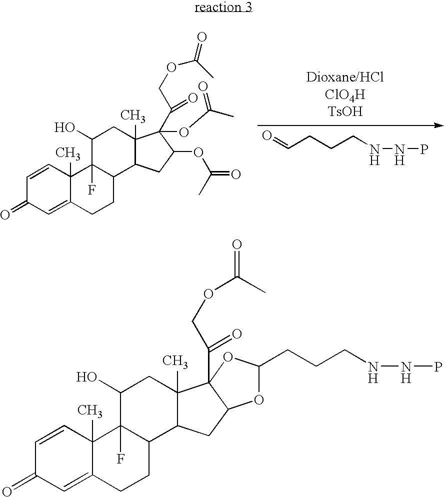

Example 5: Preparation of Hyaluronic Acid Conjugates of Triamcinolone

The protecting group in the cyclic acetal of Example 3 can be removed, vide supra, and the free hydrazine coupled to a carboxyl group of hyaluronic acid as described by, for example, Vercruysse et al., Bioconjugate Chem., 8:686, 1997 or Pouyani et al., J. Am. Chem. Soc., 116:7515, 1994. The structure of the resulting hydrazide conjugate is provided below.

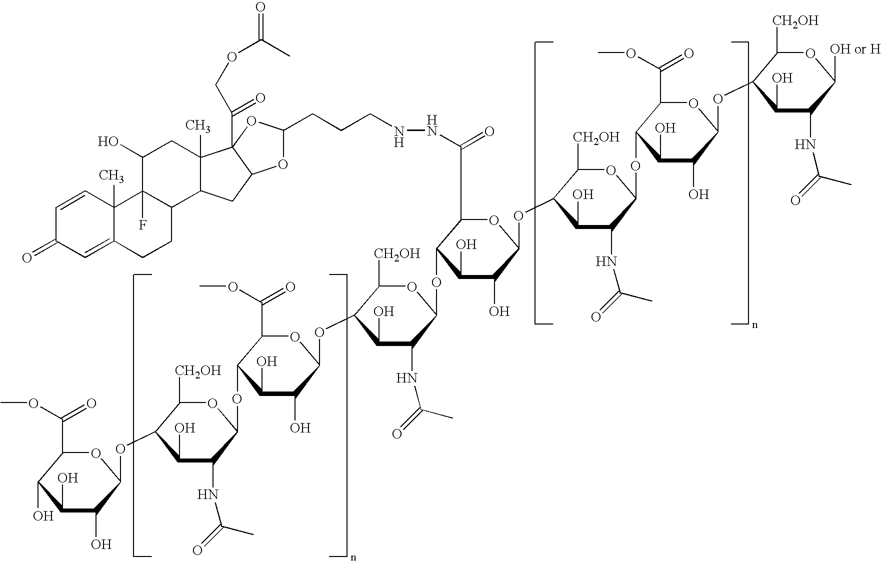

In the triamcinolone conjugate above, the hyaluronic acid is approximately 160,000 Daltons in molecular weight. Accordingly, m and n are whole integers between 0 and 400.

### Example 6

### Preparation of mPEG Conjugates of Budesonide

The cyclic acetal of budensonide can be removed in the presence of a strong acid. The resulting C16-C17 bis-hyroxyl derivative can be treated as described in Example 4 and shown in reaction 4 below.

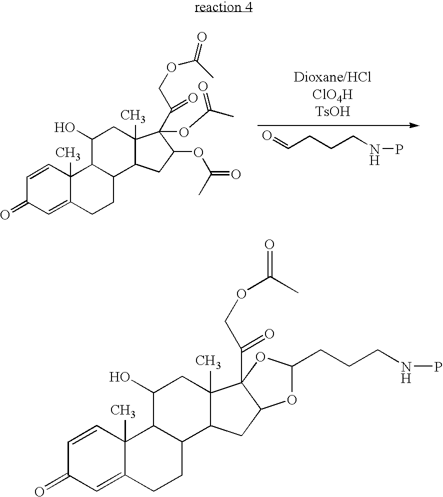

The amine protecting group can be removed and the budesonide conjugated to mono-methyl polyethylene glycol 5,000 propionic acid N-succinimidyl ester (Fluka, product number 85969). The resulting mPEG conjugate, shown below, is an example of a corticosteroid conjugate of a bulky uncharged group.

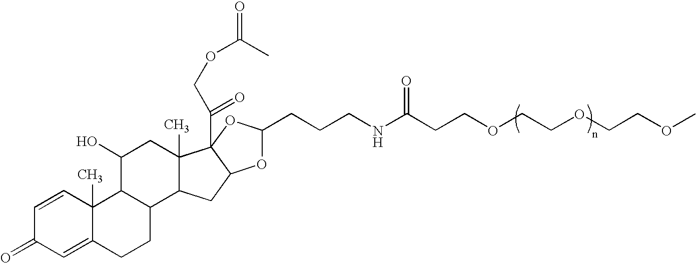

### mPEG-Budesonide, n is Approximately 110

Conjugates of lower and higher molecular weight mPEG compounds can be prepared in a similar fashion.

### Example 7

### Preparation of a Beclomethasone Dimer

21-methanesulfonate beclomethasone can be prepared according to the methods described in U.S. Pat. No. 2,932,657. The corresponding amine can be prepared by reaction with potassium phthalimide using the methods described in

### Example 2

The resulting beclomethasone amine derivative can be reacted with the bis activated ester of 1,10-decanedicarboxylic acid (Aldrich, catalogue number D100-9), as shown in reaction 5 below.

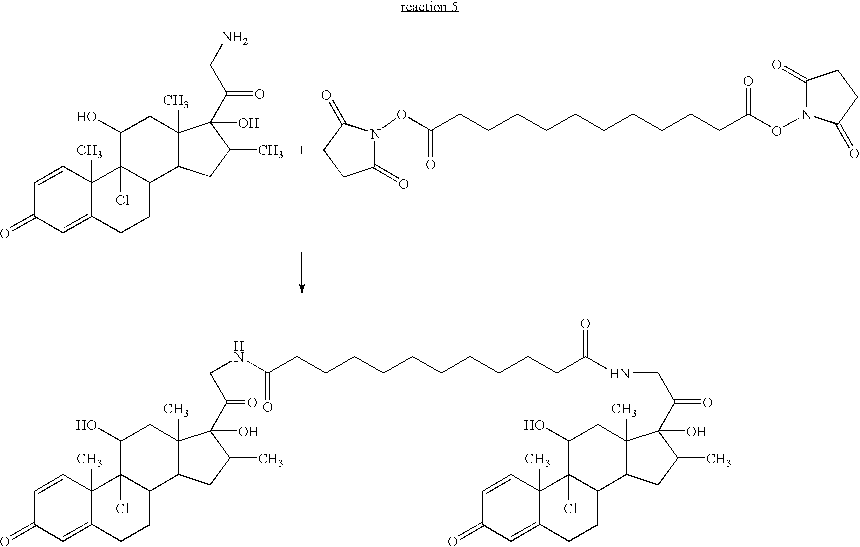

Trimers and tetramers can be prepared in a similar manner from tris-carboxyl linkers and tetra-carboxyl linkers, respectively.

### Example 8

### Preparation of a Dexamethasone-Guanidine Conjugate

21-amino dexamethasone can be prepared, for example, using the methods described in Example 2. The 21-amino group can be converted to a guanidine group. The conversion of amino groups to guanidine groups can be accomplished using standard synthetic protocols. For example, Mosher has described a general method for preparing mono-substituted guanidines by reaction of aminoiminomethanesulfonic acid with amines (Kim, K.; Lin, Y.-T.; Mosher, H. S. Tetrahedron Lett. 29:3183, 1988). A more convenient method for guanylation of primary and secondary amines was developed by Bematowicz employing 1H-pyrazole-1-carboxamidine hydrochloride; 1-H-pyrazole-1-(N,N′-bis(tert-butoxycarbonyl)carboxamidine; or 1-H-pyrazole-1-(N,N′-bis(benzyloxycarbonyl)carboxamidine. These reagents react with amines to give mono-substituted guanidines (see Bematowicz et al., J. Org. Chem. 57:2497, 1992; and Bematowicz et al., Tetrahedron Lett. 34:3389, 1993). In addition, Thioureas and S-alkyl-isothioureas have been shown to be useful intermediates in the syntheses of substituted guanidines (Poss et al., Tetrahedron Lett. 33:5933 1992). Guanylation of 21-amino dexamethasone produces the dexamethasone-guanidine conjugate shown below.

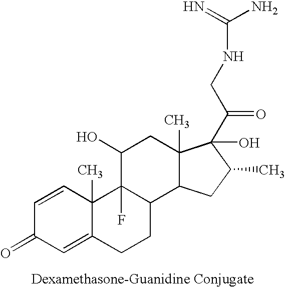

### Example 9

### Autoradiography

In vivo autoradiography can be performed using ³H-labeled corticosteroid conjugates in adrenalectomized male Sprague-Dawley rats. First, a corticosteroid conjugate is radioactively tagged and is administered systemically to an adrenalectomized male Sprague-Dawley rat, and the animal is sacrificed. The brain is then rapidly removed and sliced into 10 ˜μm thick sections and mounted on slides. The slides are apposed to tritium-sensitive film, which is developed.

### Example 10

### Displacement Binding

Displacement binding can be performed using unlabeled corticosteroid conjugates (see, Sapolsky et al., Brain Research 289:235-240 (1983)). For in vivo studies, adrenalectomized male Sprague-Dawley rats are pretreated with the varying amounts of unlabeled corticosteroid conjugate, vehicle or corticosterone. After 20 minutes, the rats are injected with radiolabeled corticosterone (1,2,6,7-3H-corticosterone; New England Nuclear) or dexamethasone (1,2,4-3H-dexamethasone; New England Nuclear) at 100 ˜μCi/100 g body weight. After 2 hours the subjects are sacrificed and the brain regions dissected on ice. Purified nuclear pellets can be prepared by centrifugation in 2 M sucrose as described by, for example, B. McEwen and A. Zigmond, “Isolation of brain cell nucleis” in Research Methods in Neurochemistry, N. Marks and R. Rodnight (eds.), New York: Plenum Press (Vol. 1), pp 140-161(1972). After ethanol extraction, fmol glucocorticoid/tissue and fmol glucorticoid/mg DNA/tissue can be calculated. In vitro cytoplasmic binding of the corticosteroid conjugate will be ascertained by dissecting hippocampi and amygdala from adrenlaectomized rats pretreated with varying doses of the conjugated compound, and then homogenizing the tissue in cold buffer. Aliquots of the cytosol will then be added to lyophilized ³H-dexamethasone. Radioactivity can be counted, and receptor Bmax can be calculated and expressed as fmol receptors bound/mg protein.

### Example 11

### Corticosterone Suppression

Plasma samples can be collected 24-hours after administration of a corticosteroid conjugate and parent corticosteroid to assay basal corticosterone levels using methods described by Lurie et al (Lurie et al., Biol. Psychiatry 26:26-34(1989)). Four hours later animals can be exposed to ether-stress, and corticosterone levels will be re-measured (Lurie et al 1989).

### Example 12

### Neurotoxicity

The neurotoxic effects of corticosteroid conjugates can be assessed using OX42 immunohistochemistry to visualize activated microglia and thereby gauge the extent of corticosterone-induced neuronal death in male Sprague-Dawley rats (Haynes et al., Neuroscience, 104:57-69 (2001)). By these methods, corticosteroid conjugates can be compared to their parent corticosteroid (on a molar basis) to assess degree of OX42 microglial response. A range of doses can be administered (typically six to eight) to establish a dose response curve for degree of observed response on silver/methenamine-stained sections.

### Example 13

### Systemic Efficacy

Samples can be incubated at 0° C. for 12 hours in the presence of unlabeled corticosteroid conjugate and 5 nM [³H] corticosterone (glucocorticoid) or [³H] aldosterone (mineralocorticoid) using methods described by Vicent et al., Mol. Pharmacol., 52:749-53 (1997). Receptors can be assayed in cytosol from thymus, fibroblasts, and kidney.

### Example 14

### Liver Assay

Using the method described by Vicent et al., Mol. Pharmacol., 52:749-53 (1997), rats can be injected in the evening prior to the experiment, and again on the morning of the experiment, with the corticosteroid conjugate, the parent corticosteroid and vehicle. After 3 hours the animals can be killed and their livers removed. Glycogen purification and quantification can be performed using the method of Krisman Anal. Biochem., 4:17-23(1962). The capacity of glucocorticoids to induce tyrosine aminotransferase (TAT) activity in hepatocytes can be measured after incubation with nM concentrations of corticosteroid conjugates, parent corticosteroids, and vehicle according to methods described by Galigniana et al., Steroids 62:358-64(1997).

### Example 15

### Thymus Assay

Male Sprague-Dawley rats can be injected with relatively large doses of a corticosteroid conjugate (equivalent to approximately 5-20 mg/kg of dexamethasone), parent corticosteroid, or vehicle. Thymus glands can be removed and weighed 72 hours later as described by Vicent et al, Mol. Pharmacol. 52:749-53 (1997)).

### Other Embodiments

All publications and patent applications, and patents mentioned in this specification are incorporated herein by reference.

While the invention has been described in connection with specific embodiments, it will be understood that it is capable of further modifications.

Other embodiments are within the claims. What we claim is:

1. A corticosteroid conjugate comprising a corticosteroid attached to a group that is either a bulky group of greater than 400 daltons or a charged group of less than 400 daltons, wherein said corticosteroid conjugate has anti-inflammatory activity in vivo and reduced activity in the central nervous system in comparison to said corticosteroid without said group.

2. The corticosteroid conjugate of claim 1, wherein said corticosteroid is covalently attached via a linker to said group.

3. The corticosteroid conjugate of claim 2 having formula I: 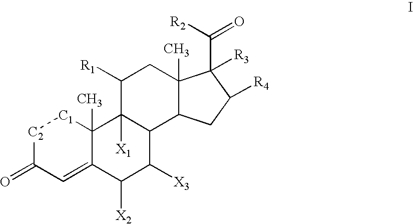
wherein
the bond between C₁ and C₂ is a double or a single bond;
X₁ represents —H or a halogen atom;
X₂ represents —H, —CH₃, or a halogen atom;
X₃ represents —H or a halogen atom;
R₁ represents ═O or —OH;
R₂ represents —CH₃, —SCH₂F, —CH₂Cl, —CH₂-G, —CH₂OH, —CH₂O—P(O)(O⁻)₂, CH₂O-acyl, —CH₂NH-G¹, —CH₂S-G¹, or —CH₂O-G¹;
R₃ and R₄ each, independently, represents —H, C₁₋₁₀ alkyl, —OH, —O-acyl, —O-G¹, or R₃ and R₄ combine to form a cyclic acetal of formula II wherein: 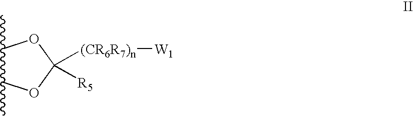
n is an integer from 0 to 6;
R₅, R₆, and R₇ each, independently, represents —H or C₁₋₁₀ alkyl;
W₁ represents —H, —CH₃, -G¹, —NR₈-G¹, —NH—NH-G¹, —O-G¹, —S-G¹, —C(O)-G¹, or —C(S)-G¹;
R₈ represents —H, C₁₋₁₀ alkyl or C₅₋₁₀ aryl; and
G¹ is a bond between said corticosteroid and said linker.

4. The corticosteroid conjugate of claim 3, wherein said linker is described by formula III:
G¹-(Z¹)ₒ-(Y¹)ᵤ-(Z²)ₛ-(R₁₀)-(Z³)ₜ-(Y²)ᵥ-(Z⁴)ₚ-G² III
wherein
G¹ is a bond between said corticosteroid and said linker;
G² is a bond between said linker and said bulky group or between said linker and said charged group;
Z¹, Z², Z³, and Z⁴ each, independently, is selected from O, S, and NR₁₁;
R₁₁ is hydrogen or a C₁₋₁₀ alkyl group;
Y¹ and Y² are each, independently, selected from carbonyl, thiocarbonyl, sulphonyl, or phosphoryl;
o, p, s, t, u, and v are each, independently, 0 or 1; and
R₁₀ is a C₁₋₁₀ alkyl, a linear or branched heteroalkyl of 1 to 10 atoms, a linear or branched C₂₋₁₀ alkene, a linear or branched C₂₋₁₀ alkyne, a C₅₋₁₀ aryl, a cyclic system of 3 to 10 atoms, —(CH₂CH₂O)_{q}CH₂CH₂— in which q is an integer of 1 to 4, or a chemical bond linking G¹-(Z¹)ₒ-(Y¹)ᵤ-(Z²)ₛ— to -(Z³)ₜ(Y²)ᵥ-(Z⁴)ₚ-G².

5. The corticosteroid conjugate of claim 1, wherein said bulky group comprises a naturally occurring polymer or a synthetic polymer.

6. The corticosteroid conjugate of claim 5, wherein said naturally occurring polymer is a glycoprotein, a polypeptide, or a polysaccharide.

7. The corticosteroid conjugate of claim 5, wherein said bulky group comprises hyaluronic acid or alpha-1-acid glycoprotein.

8. The corticosteroid conjugate of claim 5, wherein said synthetic polymer is a polyethylene glycol or N-hxg.

9. The corticosteroid conjugate of claim 1, wherein said charged group is a polyanion comprising at least three negatively charged moieties.

10. The corticosteroid conjugate of claim 1, wherein said charged group is a cation.

11. The corticosteroid conjugate of claim 1, wherein said bulky group comprises a corticosteroid.

12. A method of treating an autoimmune or inflammatory condition in a mammal, said method comprising administering to said mammal a corticosteroid conjugate of claim 1 in an amount effective to treat said condition.

13. The method of claim 12, wherein said condition is selected from the group consisting of asthma, psoriasis, eczema, organ/tissue transplant rejection, graft vs. host reactions, Raynaud's syndrome, autoimmune thyroiditis, Grave's disease, autoimmune hemolytic anemia, autoimmune thromboeytopenia purpura, mixed connective tissue disease, idiopathic Addison's disease, Sjogren's syndrome, urticaria, dermatitis, multiple sclerosis, rheumatoid arthritis, insulin-dependent diabetes mellitus, uveitis, Crohn's disease, ulcerative colitis, lupus, tendonitis, bursitis, adult respiratory distress syndrome, shock, oxygen toxicity, glomerulonephritis, vasculitis, reactive arthritis, necrotizing enterocolitis, Goodpasture's syndrome, hypersensitivity pneumonitis, glomerulonephritis; encephalomyelitis, and meningitis.

14. The method of claim 12, wherein said condition is rheumatoid arthritis or colitis.

15. The method of claim 12, wherein said corticosteroid conjugate is administered by intravenous, intraperitoneal, subcutaneous, ocular, topical, nasal, or intramuscular administration.

16. A method for inhibiting passage across the blood-brain barrier of a corticosteroid, said method comprising covalently attaching a group that is a bulky group of greater than 400 daltons or a charged group of less than 400 daltons, wherein said group increases the size, or alters the charge, of the corticosteroid sufficiently to inhibit passage across the blood-brain barrier without destroying the anti-inflammatory activity of said corticosteroid.

17. The method of claim 16, wherein said group is covalently linked via one or more of positions C16, C17, and C21 of said corticosteroid.

18. A pharmaceutical composition comprising an effective amount of a corticosteroid conjugate of claim 1, together with a pharmaceutically acceptable carrier or diluent.
